(19) United States
(12) Patent Application Publication
(10) Pub. No.: US 2002/0058329 A1
(43) Pub. Date: May 16, 2002

(54) Multiple-site reaction device and method

(76) Inventors: Sharat Singh, San Jose, CA (US); Liching Cao, Vallejo, CA (US); Herbert H. Hooper, Belmont, CA (US); David Albagli, Millbrae, CA (US); Rolfe Anderson, Saratoga, CA (US); Shulin Zeng, Sunnyvale, CA (US)

Correspondence Address: IOTA PI LAW GROUP, 350 CAMBRIDGE AVENUE SUITE 250, P O BOX 60850, PALO ALTO, CA, 94306-0850, US

(21) Appl. No.: 09/788,209

(22) Filed: Feb. 16, 2001

Related U.S. Application Data
(60) Provisional application No. 60/183,626, filed on Feb. 18, 2000.

(51) Int. Cl. C12M 1/34

(52) U.S. Cl. 435/287.2; 435/288.5; 435/6

(57) ABSTRACT

A method and device for performing a plurality of small-volume reactions simultaneously are disclosed. The device includes an elongate or planar channel and a port for introducing such bulk-phase medium into the channel, a plurality of discrete small-volume reaction regions within the channel, and a reaction-specific reagent releasably carried on a wall portion of each reaction region. In carrying out the method of the invention, a bulk phase medium containing common reactants is added to the channel. Upon release of reaction-specific reagent from the wall portions of the reaction regions, a reagent-specific reaction can occur simultaneously in each region. The channel is dimensioned to substantially prevent convective fluid flow among the reaction regions during such reactions.

This application claims the benefit of U.S. Provisonal Application No. 60/183,626 filed Feb. 18, 2000, which is incorporated herein by reference in its entirety.

### FIELD OF THE INVENTION

The present invention relates to small-volume reaction devices, and in particular to a device having a multiple-site reaction chamber in which a plurality of small-volume reactions can be carried out simultaneously, and to methods employing the device.

### BACKGROUND

The conjunction of increasing biological targets and compounds for potentially modulating the activity of the targets requires new ways to perform assays. The recognition of single nucleotide polymorphisms (“snps”) as a potential source to screen genomes for traits related to responses to drugs, susceptibility to disease, physical capacity, and the like, creates a need for methodologies to determine the snps. The increasing interest in elucidating the numerous biological pathways in plants, animals and single celled species requires improvements in the performance of numerous determinations associated with molecular interactions, such as protein-protein binding, ligand-protein binding and protein-nucleic acid binding.

As the number of operations increases, there are many reasons for wanting to be able to carry out determinations in small volumes. Small volumes offer many advantages, not the least of which are reduced amount of reagents, speed for the reactions to occur, increased number of determinations within a small area, and the reduced size of equipment in relation to the number of determinations performed. The amount of reagent is important, since many of the protein targets are only difficult and costly to produce. For candidate compounds, frequently drugs, which are increasingly coming from combinatorial libraries, the amounts available for the first screen are extremely small. With the large number of compounds produced from a combinatorial library, it is of interest to be able to run as many as possible simultaneously or at least consecutively within a short period of time. The large number of proteins present in a cell and the nature of their interactions with other naturally occurring or synthetic compounds offers a major challenge in being able to screen individual proteins against a large library of other compounds.

Toward the end of reducing volumes in which determinations are carried out, a number of investigators have reported the use of capillary electrokinesis on a small substrate, where the channels and reservoirs are of sub millimeter dimensions. These approaches tend to involve individual operations for each unit, even though there may be common reagents. In addition, the necessity for a voltage source can have a negative effect on the determination. Illustrative approaches may be found in U.S. Pat. Nos. 5,876,946; 5,872,010; and 5,922,604; and PCT applications nos. WO99/51772; 99/34920; 99/09042; 99/11373; 98/52691; and 98/00231. There is, therefore, substantial interest in developing new techniques that provide for mesoscale operations in an efficient and economical manner.

There is substantial interest in being able to perform multiple reactions in nanoliter-scale volumes simultaneously, where each of the reactions may be addressed individually. Such systems would provide for reagent savings, increased sensitivity, direct comparisons, and the like. Operations should include the polymerase chain reaction, binding, enzyme reactions, identification of nucleic acid sequences or single nucleotide polymorphisms, etc.

PCT WO99/34920 describes a platen having a plurality of through-holes as a holder for individual reaction volumes of less than 100 nl. U.S. Pat. Nos. 5,837,551; 5,834,319; 5,807,755; 5,599,720; 5,516,635; 5,4432,099; 5,304,498; and 4,745,072 are a series of patents by Roger P. Ekins of assays employing spatially separated locations. See also, U.S. Pat. No. 4,491,570. PCT/WO/98/49344 describes a method for analyzing nucleic acids with a plurality of nucleic probes as specific sites in a channel.

### SUMMARY OF THE INVENTION

The invention includes, on one aspect, a device for carrying out a plurality of different reactions in a single bulk-phase reaction medium. The device includes structure defining an elongate or planar channel and a port for introducing such bulk-phase medium into the channel, a plurality of discrete reaction regions within the channel, and a reaction-specific reagent releasably carried on a wall portion of each reaction region, for reacting in solution with one or more reagents in the bulk-phase medium, when such medium is introduced into the channel, to effect a selected solution-phase reaction in each region. The channel is dimensioned to substantially prevent convective fluid flow among the reaction regions during the reactions. The reaction regions are preferably sub-microliter in volume, e.g., 25-600 nl.

The channel preferably has a substantially uniform cross-section along its length, channel width and depth dimensions between about 20-1,000 microns, and a linear, spiral, or serpentine channel shape along its length. Alternatively, the channel may have a plurality of cross-sectionally bulged regions corresponding to the reaction regions. In another embodiment, the channel is defined by a pair of planar expanses that are spaced from one another by a distance of between 20-1,000 microns.

For use in carrying out sequence-specific nucleic acid reactions involving target nucleic acid present in the bulk-phase medium, the reaction-specific reagents are nucleic acid oligomer reagents releasably bound to the wall portions, e.g., through duplex formation with immobilized complementary-sequence oligonucleotides, or via ligand attachment to an immobilized antiligand. For example, each reaction region may include a capture nucleic acid immobilized on the associated wall portion and having a region-specific nucleic acid sequence, wherein different-sequence nucleic acid oligomer reagents are hybridized with such capture nucleic acids.

The device having nucleic acid reaction reagents may be used, for example, for (a) polymerase extension reactions, where the reaction-specific reagents in each region include extension primers; (b) PCR reactions in the reaction regions, where the reaction-specific reagents in each region include one or more sets of PCR primers, or (c) sequence-specific 5′ exonuclease reactions that result in the formation of a detectable product, where the reaction-specific reagent in each region include as an exonuclease substrate, an oligonucleotide having a selected nucleic acid sequence terminating in a detectably labeled 5′ nucleotide. For use in carrying out 5′ exonuclease reactions, detectably labeled 5′ nucleotides associated with different reaction regions are electrophoretically separable.

In a more specific aspect, the invention includes a device for carrying out simultaneous sequence-specific nucleic acid reactions on a plurality of DNA target segments (i) contained in a bulk-phase medium and (ii) having different nucleic acid sequences. The device includes a substrate defining an elongate or planar channel terminating at first and second ends, a lid covering the open channel to form an elongate closed channel terminating at first and second ports, a plurality of discrete reaction regions spaced along the length of said channel, between said ports, and in each reaction region, one or more region-specific nucleic acids releasably carried on a portion of that reaction region.

The region-specific nucleic acids are effective to bind to complementary sequence nucleic acid target segments contained in the bulk-phase medium, after such medium is introduced into the channel and the channel is dimensioned to substantially prevent convective fluid flow among the reaction regions in the channel, whereby the region-specific nucleic acids are largely confined to the associated region during such reaction.

The device has specific features as mentioned above. The substrate may be designed to be placed in a centrifugation apparatus, such that centrifugation of the device is effective to cause liquid medium introduced at one port to fill the channel, or liquid medium contained within the channel to be expelled therefrom. Reaction product may be captured in each reaction region on capture nucleic acids immobilized on the channel wall portions.

The invention also contemplates a card having a plurality of such devices, each providing an elongate channel for carrying out multiple simultaneous reactions. The card may have various channel and port configurations to facilitate simultaneous loading and unloading of bulk-phase sample material from the devices in the card.

In another aspect, the invention includes a method for simultaneously carrying out a plurality of different reactions that involve both common and reaction-specific reagents. The method includes the steps of (a) filling a channel in a device of the type described above with a bulk-phase medium and reagents common to the plurality of reactions, (b) providing reaction-specific reagents to the individual reaction regions, and (c) simultaneously promoting reactions involving reagents provided in the bulk phase and the reaction-specific reagents in each of the reaction regions.

After completing the reactions, the medium may be removed from the device for analysis or processing of the plurality of reaction products. The reaction-specific reagents may be supplied by adding such reagents through ports accessing discrete regions along the length of the channel, or may be released from channel wall portions of the separate reaction regions.

The device may be used for carrying out simultaneous PCR reactions on a plurality of different DNA targets contained in the bulk-phase medium. Here the reaction-specific reagents in the different reaction regions include PCR primers designed to hybridize with and amplify different, selected regions of the DNA targets. The PCR reactions are promoted by successively heating and cooling the device, under conditions effective to produce PCR amplicons.

In still another aspect, the invention includes a method for carrying out a plurality of simultaneous sequence-specific nucleic acid reactions on a plurality of DNA target segments (i) contained in a bulk-phase medium and (ii) having different nucleic acid sequences. The method includes adding to a device having (i) structure defining an elongate channel and a port for introducing a liquid medium into the channel, and (ii) region-specific capture nucleic acids immobilized on channel wall portions at a plurality of discrete reaction regions contained within and along the length of the channel, a solution containing a plurality of different-sequence nucleic acid reagents. Each reagent has a capture portion effective to hybridize to one of the capture nucleic acids and a reaction portion effective to hybridize to one of the target DNA sequences in the bulk phase medium, under DNA hybridization conditions. This step is effective to localize selected nucleic acid reagents at selected reactions regions in the channel. After filling the channel with the bulk phase medium, reactions involving target segments contained in the bulk phase medium and such region-specific nucleic acid reagents are promoted by causing release of the nucleic acid reagents from the associated reaction-region wall portions.

In a related aspect, the invention includes carrying out a small-volume nucleic acid reaction by adding to a small-volume reaction region, a bulk-phase medium containing reaction reactants. The wall portion of the region has immobilized capture nucleic acids to which are releasably bound, by sequence-specific hybridization, one or more oligo- or poly-nucleotides that participate in the reaction, e.g., PCR reaction. After carrying out the reaction, the reaction product, e.g., amplified DNA segments, are captured in the reaction region by hybridization to the immobilized capture nucleic acids. The region may then be washed to remove unbound reagents, and the product either detected in situ or released in concentrated form.

In another aspect, the invention includes a method for performing a plurality of affinity determinations to determine the biological activity of candidate compounds employing an elongated channel having a cross-section in the range of about 10 um² to about 4 mm² and a plurality of sites at which are non-diffusively bound a first component of said affinity determination. Each site is bordered by a source trench and a drain trench for moving components of the affinity determination to and away from the site. The affinity determination comprises first binding a candidate compound to an enzyme and employing an enzyme substrate which results in a detectable product.

The method includes the steps of electrokinetically moving each of said candidate compounds from each of the source trenches to each of their respective sites and incubating the resulting mixture at each site, resulting in a detectable product, adding substrate to the main channel, electrophoretically moving the detectable product from the site to the drain trench, and detecting the detectable product separate from other components of said affinity determination as a measure of said affinity determination. The length of the site and the cross-section of the channel are chosen to have a reaction volume for said affinity determination of less than about 100 nL.

These and other objects and features of the invention will become more fully apparent when the following detailed description of the invention is read in conjunction with the accompanying drawings.

### BRIEF DESCRIPTION OF THE DRAWINGS

FIGS. 1A and 1B are plan and sectional views of a microfluidics device constructed in accordance with one embodiment of the present invention;

FIGS. 2A and 2B are plan and sectional views of a microfluidics device constructed in accordance with another embodiment of the invention;

FIGS. 3A and 3B are plan and sectional views of a microfluidics device constructed in accordance with a third embodiment of the invention;

FIG. 4 shows a device like that in FIG. 1, but having for each channel region, a pair of side channels through which solute or solution material can be added to or removed from the associated reaction region;

FIG. 5 shows a card with a plurality of reaction devices formed therein;

FIGS. 6A and 6B show steps in introducing fluid into one of the channels in the FIG. 5 device;

FIGS. 7A-7D illustrate exemplary methods for removing liquid from a channel in the FIG. 5 device;

FIGS. 8A-8D show steps in introducing fluid into and removing fluid from one of the channels in another embodiment of a card device in accordance with the invention;

FIGS. 9A-9C show alternative methods for releasably binding reaction-specific reagents, e.g., nucleic acids to the wall portion of a reaction region in the device of the invention;

FIG. 10 shows three adjacent wall portions in a channel, in accordance with the invention, illustrating three different-sequence nucleic acid primers releasably immobilized to the reaction-site wall portions through site-specific nucleic acids immobilized on the wall portions of the three sites;

FIGS. 11A-11E illustrate steps in carrying out simultaneous PCR reactions in accordance with the invention; and

FIGS. 12A-12C illustrate steps in carrying out simultaneous PCR reactions in accordance with the invention.

### DETAILED DESCRIPTION OF THE INVENTION

I. Definitions

Unless otherwise indicated, the terms below have the following definitions herein.

An “elongate channel” is a substantially one-dimensional channel having a length dimension that is at least 1-2 orders of magnitude greater than the width dimension of the channel. The channel may be linear or curved, e.g., spiral or serpentine. The channel has preferred width and depth dimensions between 20-1,000 microns, typically 25-500 microns, and a length of up several cm's or more. A channel having these depth and width dimensions is also referred to herein as an elongate microchannel.

A “planar channel” is a sheetlike channel formed between two closely spaced planar expanses, e.g., plates whose confronting surfaces are spaced 20-1,000 microns, typically 50-500 microns from one another. A channel having these between-plate spacings is also referred to herein as a planar microchannel.

A “bulk-phase reaction medium” is an aqueous solution containing one or more reagents that are common to different reactions carried out in the device of the invention. For example, for carrying out PCR reactions in the device, the bulk-phase medium will typically contain target DNA to be amplified, DNA polymerase, all four nucleotide triphosphates and other components needed, in combination with reagent(s) supplied in each reaction region, e.g., DNA primers, for carrying out the desired reaction.

A channel is “dimensioned to substantially prevent convective flow” if the spacing between confronting walls of the channel (either elongate or planar) are such as to limit the mixing of solute molecules within the channel to diffusional mixing, as opposed to convective mixing within the bulk phase. Channels having width and depth dimensions in the 20-1,000 micron, preferably 50-500 micron size range and planar channels having between-plate spacing in the same dimension ranges are so dimensioned.

“Small-volume reaction regions” refers to reaction regions having volumes of about 1 microliter or less, typically 25-600 nanoliter.

“Discrete reaction regions” means that at least some reaction regions are spaced one from another in a channel. Preferably, each reaction region is spaced apart from all other regions in the channel.

A “sequence-specific nucleic acid reaction” is one that occurs only when a target DNA reactant contains a specific sequence. Such reactions include, without limitation, primer-initiated polymerization or ligase reactions, polymerase chain reaction (PCR), primer-dependent 5′-exonuclease reactions, and restriction endonuclease reactions.

“Region-specific nucleic acids” refers to oligonucleotide or polynucleotide molecules that have a selected sequence or region of sequence that is different for different reaction sites, thus allowing different sequence-dependent reactions to occur in the different reaction regions of the device of the invention.

“Releasably bound”, as applied to one or more reagents, means that the reagent(s) remain bound to the wall portion, when a bulk-phase medium is introduced into a reaction site, but are released into the bulk phase medium either passively over time, or actively by the application of heat, light or other external stimulus, or by the inclusion in the bulk phase of specific cleavage agents, such as a reducing agent or hydrolytic enzymes. As used herein, the term is synonymous with “releasably and non-diffusively bound”, where a reagent is non-diffusably bound if it is not released from a reaction-region wall portion upon initial hydration with bulk-phase medium.

II. Multisite Reaction Device

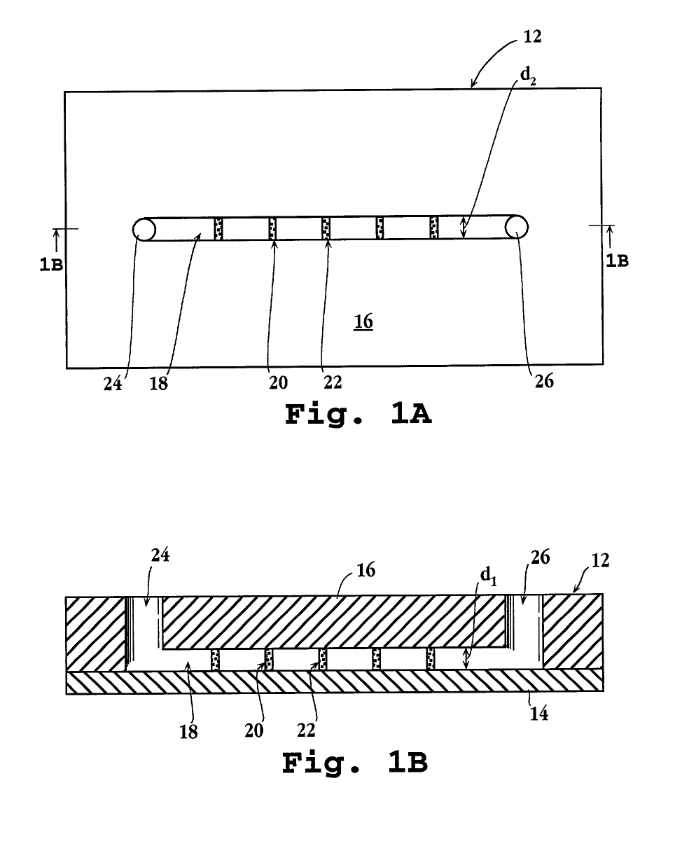

FIGS. 1A and 1B are plan and sectional views, respectively, of a device 12 constructed according to an embodiment of the invention, for carrying out a plurality of different reactions in a single bulk-phase reaction medium. The device includes a substrate 14 and a covering 16 which is attached, as by thermal welding or the like to the substrate. Formed in the covering is a channel 18 extending between an input port 24 and an output port 26. As can be appreciated, the substrate serves to enclose the channel, confining liquid movement within the channel through ports 24, 26. Alternatively, the channel may be formed in the substrate and enclosed by the covering over the substrate. The substrate and covering thus provide means defining an elongate channel in the device. Other channel-defining means can include a tube, such as a capillary tube, an integral molded structure with an internal microchannel.

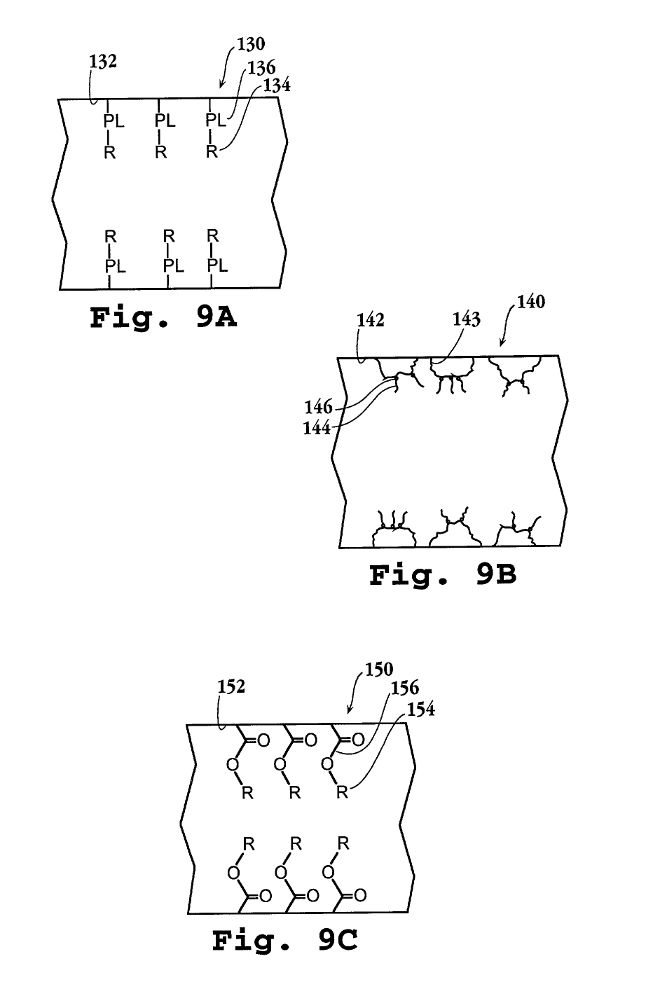

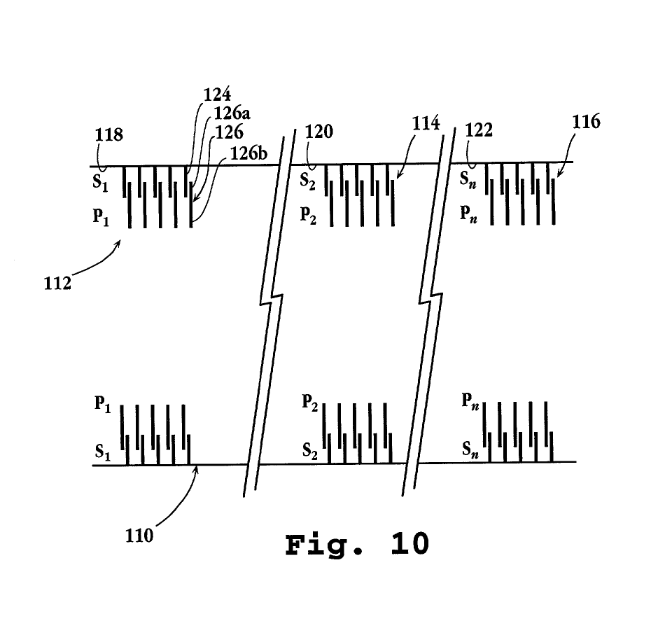

According to an important aspect of the invention, the device includes a plurality of discrete reactions regions, such as regions 20, 22, within the channel, at spaced positions along the length of the channel. The portion of the channel extending through the reaction regions has a wall portion, such as the top or side channel wall portions formed in covering 16, to which reaction-specific reagent(s) are releasably attached. As will be considered below with reference to FIGS. 9 and 10, the reagent(s) are released after bulk-phase medium is introduced into the channel, providing reactant(s) that are specific for each reaction site. The reagent(s) react in solution with reactants contained in the bulk-phase (and thus present at all reaction sites) in a reaction that is site specific, that is, determined by the reagent(s) released in each site.

The channel is dimensioned in width and depth to substantially prevent convective fluid flow between adjacent reaction sites. That is, to the extent reactants in each reaction site are able to mix over the course of the reaction carried out in each site, such mixing occurs primarily by diffusion of solute components rather than by bulk-phase stirring by convection. This feature limits the spread of solute reaction components, including reaction products, to that site and, at most, adjacent sites.

To this end, the channel is generally of a cross-sectional area of not more than about 1 mm², usually less than about 0.8 mm², preferably less than about 0.4 mm², and frequently as small as about 50μ² or in some situations, may even be less. The cross-section may be circular or non-circular. For non-circular cross-sections the channels will generally have an average depth of about 5μ to 1 mm, preferably in the range of about 5 to 500μ, more usually 100 to 300μ, and an average width in the range of about 10μ to 1 mm, more usually 25 to 500μ. Selection of the size of the channel will depend on the reaction volume desired, the nature of the signal to be detected, the sensitivity of the detection system, and the like.

The length of the channel will usually be at least about 0.5 cm, usually at least about 1 cm, and may be 20 cm or more, usually not more than about 10 cm. The length will be, to a degree, dependent on the number of reaction regions, the length of the individual regions, and the separation between regions. Although a linear channel is shown, it will be appreciated that other elongate channel configurations are possible, e.g., a serpentine or spiral channel, and these more compact channel shapes will generally be desirable when the device is constructed in microchip form, e.g., on a surface having an area of 1 cm² or less.

Desirably, the reaction volume of each reaction region will be in the range of about 5 nl to 900 nl, usually in the range of about 5 nl to 600 nl, more usually in the range of about 10 nl to 300 nl. By reaction volume is intended the region of the channel in which reaction is performed. The length of the area of the specific binding member will generally be in the range of about 10 nm to 5 cm, more usually 100 nm to 2.5 cm, frequently 10 microns to 10 mm, depending on the purpose of the operation and the required capacity for binding.

The substrate in which the capillary channels are formed may be of any convenient material, such as glass, plastic, silicon, or the like. Various plastic or organic polymeric materials include addition and condensation polymers and copolymers, linear or cross-linked, clear, semi-translucent, or opaque, mixtures of polymers, laminates and combinations thereof. Polymeric materials include polyethylene, polypropylene, acrylics, e.g. poly(methyl methacrylate), polycarbonate, poly(vinyl ethers), polyurethanes, dimethyl siloxanes, poly(4-methylpentene-1), etc. Desirably the polymers should be capable of extrusion or molding. Where the reaction sites are viewed directly, i.e., in situ, the covering in the device must be optically clear at the detection wavelengths employed.

Methods of fabricating channels in such substrates, and welding substrate and covering components are well known in the microfabrication field. It should be noted that localized or low-temperature welding techniques must be employed where the channel regions are initially loaded with a heat-sensitive biological material, such as a biological polymer or heat unstable binding agent. To this end, a variety of adhesives or techniques for surface-localized thermal binding are available, such as ultrasonic welding or laser welding.

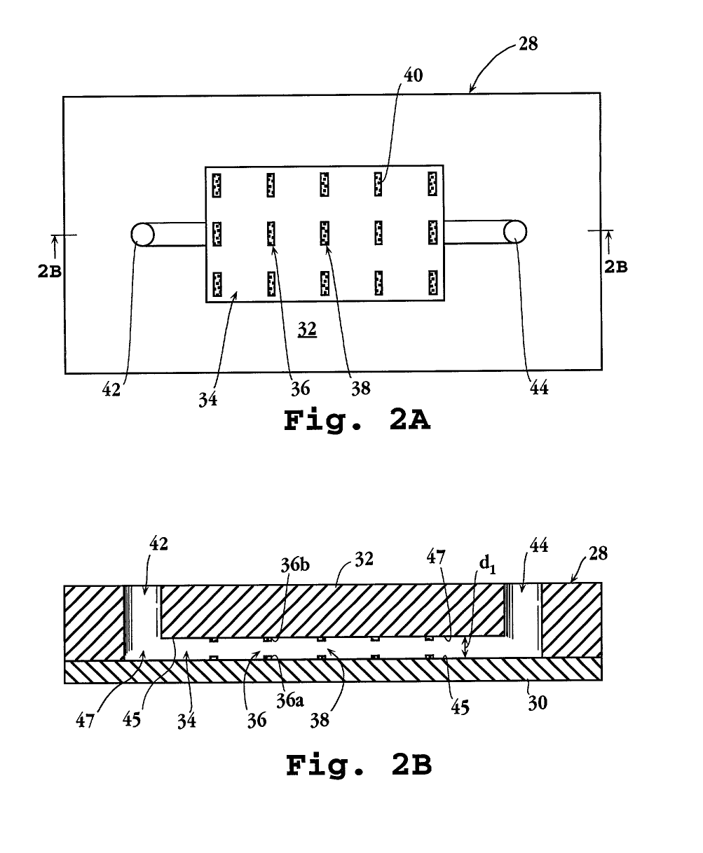

FIGS. 2A and 2B are plan and sectional views, respectively of a multi-site reaction device 28 constructed in accordance with another embodiment of the invention. The device includes a substrate 30 and covering 32 which together, form a planar channel 34 in communication with input and output ports 42, 44, respectively. That is, the channel is a thin planar expanse formed between confronting surfaces 45, 47 of the substrate and covering, respectively. Bulk-phase liquid is moved in and out of the channel through the two ports.

As seen particularly in FIG. 2A, the planar channels includes a plurality of discrete reaction regions, such as regions 36, 38, 40 which are arranged in a two-dimension array of sites within the channel. Each reaction region, such as region 36, is defined by upper and lower wall portions, such as wall portions 36a, 36b, having a reaction-specific reagent releasably bound thereto, for release in the reaction region between the two wall portions, when bulk-phase medium is added to the channel. Exemplary modes of releasably binding reagents to a reaction site wall portion are discussed below with reference to FIGS. 9 and 10.

The distance d₁ between the confronting channel surfaces is between about 20-1,000 microns, preferably 50-500 microns. In particular, the channel thickness is dimensioned to substantially prevent convective fluid flow among the reaction regions when a bulk-phase liquid is introduced into the channel. In addition, the channel may be provided by porous barriers, not shown, that act to limit lateral convective flow. Such barriers may, for example, effectively partition the planar channel into a plurality of elongate subchannels, such as the subchannel aligned with ports 42, 44, and containing reaction regions 36, 38, where the distance between adjacent barriers is, for example, comparable to the channel width dimension in device 12.

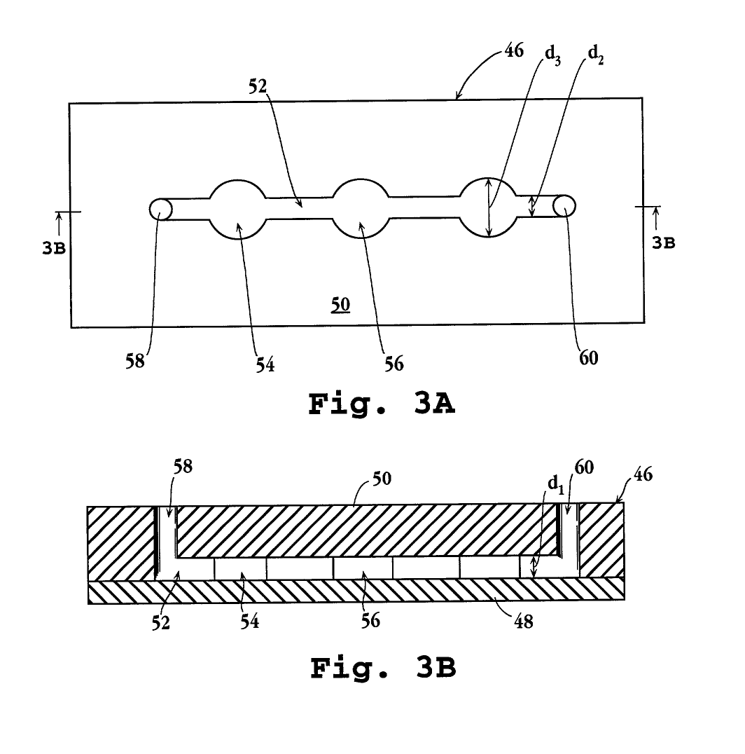

FIGS. 3A and 3B are plan and sectional views, respectively, of a multi-site reaction device 46 constructed according to another embodiment of the invention. The device is formed of a substrate 48 and covering 50 which together define a closed elongate channel 52 connected at its opposite ends to ports 58, 60, similar to device 12. The device differs from device 12 in that the reaction regions, such as regions 54, 56, formed within and along the length of channel 52, are radially enlarged, as seen in FIG. 3B. Preferably the reaction regions are shaped as in FIG. 3 to promote efficient removal of reaction-region components, e.g., products, from the device upon completion of the reactions in the device. The reaction sites contain reaction-specific reagent(s) releasably bound to wall portions of the regions, as above.

The depth d₁ and width d₂ dimensions in the device are similar to those in device 12, that is, preferably between 20 and 1,000 microns, more preferably between 50-500 microns. The lateral dimension d₃ of each reaction region is typically 1.5-3 times that of width d₂. This configuration has the advantage over device 12 in providing greater-volume reaction regions while still limiting convective flow between the regions through the narrowed connecting channel portions.

FIG. 4 is a plan view of a multi-site reaction device 62 constructed according to still another embodiment of the invention. The device includes a substrate (not seen) and covering 64 which define an elongate channel 66 communicating at its opposite ends with ports 68, 70, similar to the construction of device 12 above. Contained within the channel, at sites spaced therealong, are a plurality of reaction regions, such as regions 72, 74, each having reaction-specific reagent(s) releasably bound to a wall portion in each region, also as described above.

In addition, the device provides, for each reaction region, a pair of side channels, such as side channels 76, 78 associated with region 72, for adding material to the associated reaction region, from one of the side channels, and/or removing material from the reaction region from the other side channel. Each channel is connected at its distal end to a reservoir, such as reservoir 80 connected to channel 76, for containing a buffer or reagent solution. The reservoirs may be provided with electrodes by which an electric field can be placed across the associated reaction region, for moving material into or out of the region by electrokinetic movement, e.g., electroosmotic flow or electrophoretic movement of charged solute molecules. Alternatively, the device may be designed and operated to move solution from the side channels in or out of associated reaction material by a pressure gradient.

FIG. 5 illustrates a microfluidics card 80 which is formed to include a plurality of multi-site reaction site devices, such as devices 82, 84 of the type described above. Specifically, each device includes an elongate channel, such as channel 86 in device 82, and each channel includes a plurality of reaction regions within the channel and spaced along the length of the channel. The card illustrated, which includes and 8×12 array of devices, is designed for use in carrying out groups of up 96 simultaneous reactions, e.g., PCR reactions.

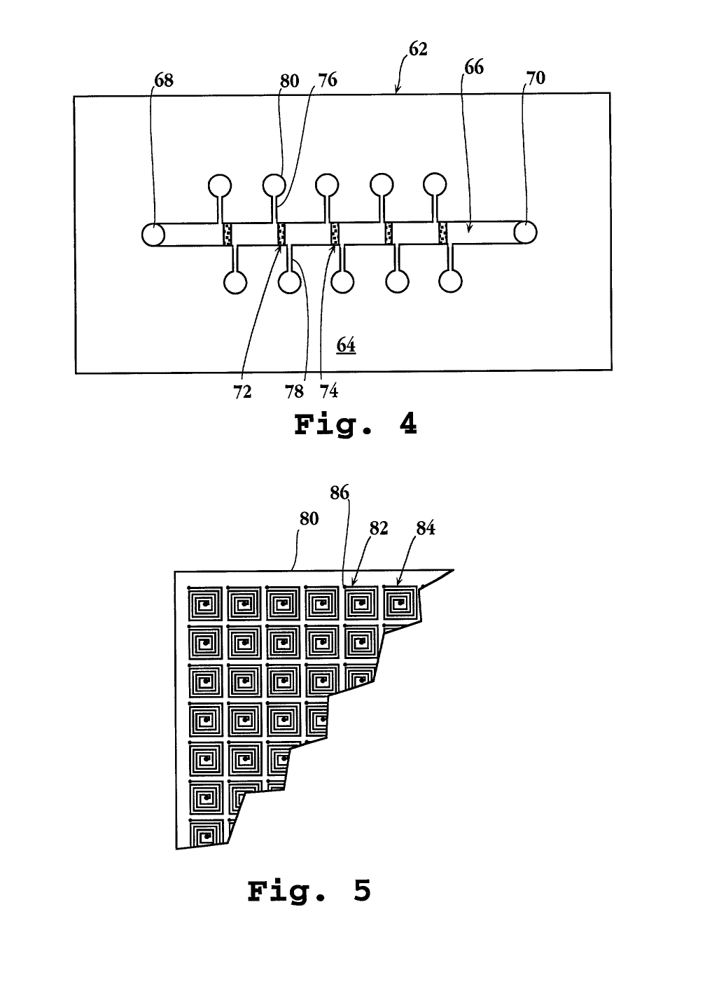

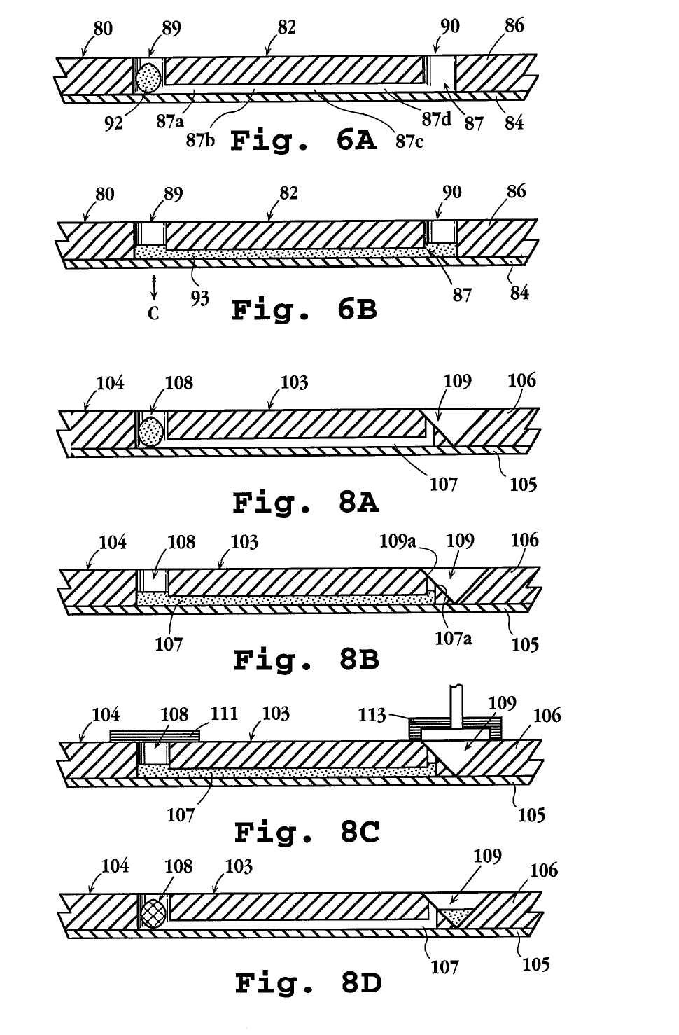

The construction of device 82 in card 80 is seen cross-sectionally in FIGS. 6A and 6B. The card includes a substrate 84 and a covering 86 which together define the spiral channel of each of the several devices in the card. Device 82, which is representative includes elongate serpentine channel 87 having inlet and outlet ports 88, 90 at opposite ends of the channel, and a plurality of reactions regions, such as regions 87a, 87b, 87c, and 87d within and along the channel. As above, each of the reaction regions carries reaction-specific reagent(s) releasably bound to the wall portion of that region. (The channel is shown in linear form in FIGS. 6-8, it being recognized that the inlet port is at one corner of the device, and the outlet port, at the center of the device, as in FIG. 5).

In FIG. 6A, a drop 92 of bulk-phase medium is placed in port 89 (and in the ports of other devices on the card). The card is placed in the bucket of a centrifuge subject to a centripetal force in the direction of arrow C, forcing the liquid droplet through the channel, as illustrated in FIG. 6B. The movement of liquid under the centrifugal field is self-limiting once a common liquid level is reached through the channel, since there is no longer a driving force on the liquid at this point. The sheet of bulk-phase liquid in the channel is indicated at 93 in FIG. 6B.

After carrying out the multiple simultaneous reactions in each device of the card, e.g., by successive heating and cooling in the case of a PCR reaction, the liquid in the device channels is removed for product analysis. Several liquid-retrieval methods are illustrated in FIGS. 7A-7D. In the method illustrated in FIG. 7A, the substrate is punctured, as at 94, at each of the device outlet ports, such as port 90 in device 80, and a capture plate 96 is placed against the substrate. The capture plate has a plurality of wells, such as well 97 which are arrayed on the plate for registration with corresponding outlet ports in the card devices. The card and capture plate are then centrifuged so as generate a force in the direction of arrow C in FIG. 7A, to drive liquid in each channel in the card into a corresponding well in the capture plate. The liquid samples in each well can then be individually handled by conventional microtiter plate methods.

Alternatively, and with reference to FIG. 7B, a capture plate 98 having wells, such as wells 99, 100 corresponding to the two ports in each device may be placed against the covering in the device, that is, with the device inverted. Centrifugation in the direction generating a force C then drives the liquid from each device into the two wells of the capture plate.

Yet another liquid-retrieval approach is illustrated in FIGS. 7C and 7D. In this method, a droplet, such as droplet 102, of a liquid more dense than the bulk-phase solution in the each channel is placed in the inlet port of each device, such as port 89 of device 82. The card is then centrifuged with a force in the direction of arrow C, causing the heavier liquid to displace the bulk-phase liquid in the channel and drive the sample liquid into the outlet port of each device, such as port 90. The sample can then be analyzed and/or removed according to standard microtiter plate methods.

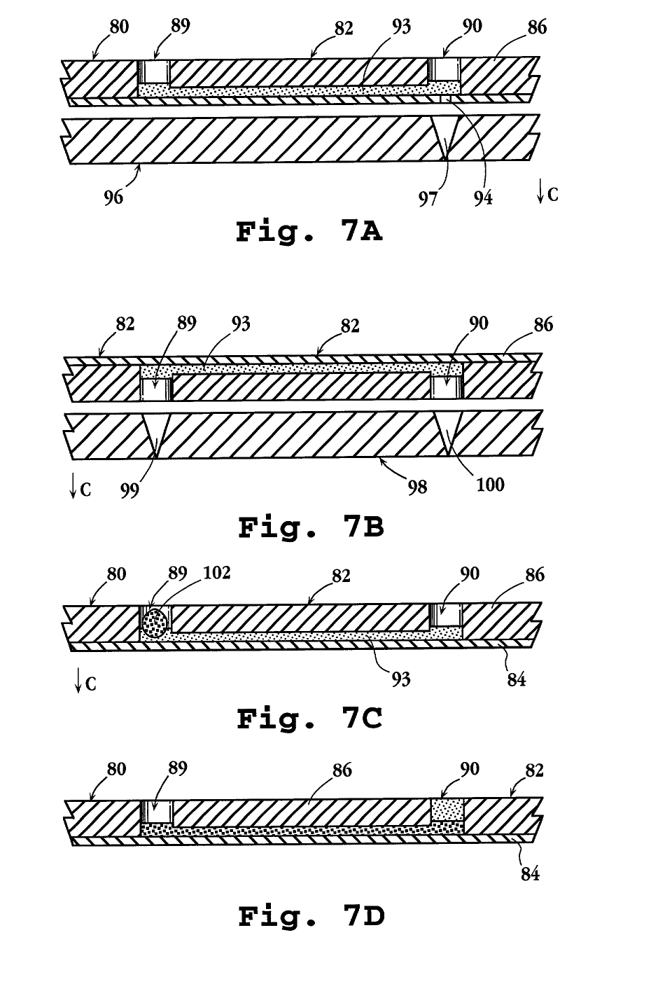

FIGS. 8A-8D show an alternative construction of the devices, such as device 103 in a multi-device card 104. The card has the general construction of that described above, being formed of a substrate 105 and a covering 106 defining, and defining a plurality of multi-site reaction devices, such as device 103, in the card. Device 103, which is representative, includes an elongate spiral channel 107, having a plurality of reaction regions formed within the channel and spaced along its length, and inlet and outlet ports 108, 109, respectively. As seen in the figures, outlet port 109 communicates with and “upwardly” directed end portion 107a of the channel along a angled wall portion 109a thereof, such that the channel empties into an upper or distal portion of the port.

In operation, a drop of bulk-phase medium is placed in the inlet port of each device, such as port 108 in device 103, and the card is centrifuged, as described above, to force liquid into the channel, as in FIG. 8B. After carrying out multiple reactions in each of the loaded devices, bulk-phase medium is retrieved by (i) placing a seal 111 over each inlet port, such as inlet port 108 in device 103, and a suction device 113 over each outlet port, to draw liquid out of each channel and into the associated outlet port, as illustrated in FIGS. 8C and 8D, producing a sample of bulk-phase medium in each outlet port. The sample can then be handled according to standard microtiter plate procedures.

It will be appreciated that the various methods just described for introducing bulk-phase medium into a device channel, and removing it therefrom are also applicable to reaction system having a single device of the type described with respect to FIGS. 1-4.

As noted above, each reaction region in the device of the invention has reaction-specific reagents(s) releasably bound to a wall portion that defines the region. By this is meant the reagent(s) remain anchored to the reaction site walls upon introduction of bulk-phase medium into the channel, but are released passively or actively thereafter, to participate in solution phase with a reaction in the reaction site.

The reagent may be any compound capable of participating in a biological of chemical reaction, and in particular, capable of reacting with one or more reactants in a bulk-phase medium to produce a reaction that is unique to the reaction region which contains the reagent. Thus, for example, the reagent may be one of a number of different binding agents or drugs, some or all of which are capable of interacting with a receptor carried in the bulk-phase solution, or one of a number of different enzyme substrates, some or all of which are capable of interacting with an enzyme contained in the bulk phase solution, or conversely, one of a number of different proteins or other enzymic or binding agents, some or all of which are capable of reacting with a given substrate or binding agent in the bulk-phase medium. In one preferred embodiment, detailed below, the reagent includes one or more oligo- or poly-nucleotides having a reaction-specific nucleic acid sequence effective to produce a sequence-specific reaction, such as one involving complementary strand hybridization or sequence-specific endonuclease cutting.

Desirably at each site there will be at least about 10 aftomoles, preferably at least about 1 femtomole, usually at least about 1 picomole and not more than about 1 millimole, more frequently not more than about 0.5 millimole of a specific binding pair member. The amount of the releasable reagent will depend upon the nature of the reaction, the specificity of the reaction, the signal produced, the sensitivity of the detection system, and the volume of the reaction region.

Depending on the nature of the surface of the channel, proteins or other substances may bind non-covalently and be stably bound during the operation. For example, methylated proteins strongly adhere to surfaces. The protein also serves to minimize non-specific binding of components of the operation. Alternatively, the reagent may be embedded in a wall coating, such as a hydrogel wall coating, or other coating material that hydrates or dissolves over a time period that is substantially greater than the time period needed to fill the channel in the device. Polymer coatings capable of holding and releasing reagents over time are also suitable for certain reagents.

Several methods of reagent binding for active release are also available. FIG. 9A shows a reaction region 130 with wall portion 132. Reagent molecules 134 are releasably bound to the wall portion by a linker covalently attached to the wall portion, and containing a photolytic group 136 that is cleaved by irradiation with a selected wavelength light, e.g., UV light. The design and synthesis of bifunctional reagents containing an internal photolytic group and capable of covalent attachment to active wall-portion functionalities, such as carboxy, amino, hydroxy or thiol groups, and to suitable reagent molecules are well known to those in the art. The reagent molecules are actively released, after addition of bulk-phase solution to the channel, by irradiating the channel with light of a photolytic wavelength.

In the method illustrated in FIG. 9B, the wall portion 142 in device 140 is covalently derivatized with streptavidin molecules 143, using well-known methods. A biotinylated reagent 144, such as biotinylated nucleic acid, is bound to the streptavidin through biotin groups, such as 146 attached to the reagent. If lower affinity binding is needed, the streptavidin may be replaced by lower-affinity binding agents, such as antibodies or receptors, and the biotin, by lower-affinity ligands, such as antigen or receptors binding agents. Release of the reagent from the wall portion can be effected, after introducing the bulk-phase medium, by application of heat or sonic energy, or another ligand that has a higher affinity for the binding agent.

FIG. 9C illustrates reagent binding through an enzyme cleavable linkage, in this case, an esterase. The figure shows a segment of a device 150 having a wall portion 152 and reagent molecules 154 covalently attached to the wall portion through ester linkages 156. Inclusion of an esterase in the bulk-phase medium, leads to slow passive release of reagent into the solution phase in the reaction region. Alternatively, in a device like the one shown in FIG. 4, the cleaving enzyme can be introduced into each reaction region from one of associated side channels, to actively release the reagent. Where the reagent is an oligo- or polynucleotide covalent bound the wall portion, the reagent may include a restriction-endonuclease site, for release from the wall portion by including the appropriate endonuclease.

In each of the attachment schemes described above, the site-specific reagent is attached to the wall portion by a common linkage or attachment to an immobilized molecule. That is, the linkage itself is common to all of the reaction regions. In this general case, the specific reagents must be added directly to specific reaction regions, either before the channel is covered or, in the FIG. 4 embodiment, by using the side channels to deliver a specific reaction region to each associated region.

In still another embodiment, the reagents are non-releasably bound to the reaction region wall portions, e.g., by covalent binding, and are employed in the reaction in immobilized form, e.g., immobilized nucleic acid primers sued in a DNA sequence reaction.

In another general case, and in accordance with one aspect of the invention, the particular reaction-specific reagents are designed to react with and bind to immobilized molecules that are unique to each reaction site. By this method, the device can be “pregrammed” with the releasable reagents simply by adding a mixture of the reagents to the channel, and allowing each reaction-specific reagent to bind to its binding pair in a selected reaction region.

The latter method is illustrated in FIG. 10, which shows three reaction regions 112, 114, and 116 in the channel of a device 110, where the corresponding wall portions are indicated at 118, 120, and 122, respectively. Covalently attached to each wall portion is a unique (site-specific) capture nucleic acid, such as oligonucleotide 124 (S₁) attached to wall portion 118, and oligonucleotides S₂ and S₃ attached to wall portions 120, 122, respectively. The capture nucleic acids in the different region are preferably at least about 7-10 bases long, typically 12 bases or more, and differ from one another in sequence by at least one, and preferably two or more bases. The capture reagent may, in addition, contain more than one capture sequence, allowing different-sequence reagents to be captured on a single capture nucleic acid. The reagent itself, such as reagent 126 (P₁) in reaction region 112, has a capture portion 126a that is complementary in base sequence to the capture nucleic acid, and a reaction portion 126b which is effective to participate in the solution-phase reaction in the reaction region. Similarly, each of the reagents P2 and P3 in regions 114, 116, respectively, has a capture portion that hybridizes to capture nucleic acids S₂ and S₃, respectively, and a reaction portion that is unique to that reaction region.

In preparing the device with the different nucleic acid reagents, a bulk-phase medium containing a mixture of the reagents is circulated through the channel under hybridization conditions, for a period sufficient to saturate the capture nucleic acids in each reaction region with the different-sequence reagents.

III. Multiple-site reaction method

The invention may be used with various protocols involving nucleic acid sequencing, nucleic acid hybridization, and the like, single nucleotide polymorphism (snp) detection, proteomics (protein-protein interactions), specific binding pair reaction (ligand-receptor), enzyme reactions, and the like. More generally, the invention may be used for any system that permits multiple reactions involving one or more common reactants, supplied to each reaction region in a bulk-phase medium, and one or more reaction-specific reagents that are supplied by each individual region region.

In performing the operations, the temperature of the regions may be varied, by heating and cooling, using heating elements in contact with the region, infra-red sources or other sources of electromagnetic radiation, the pressure may be varied, the regions may be irradiated with light in the wavelength range of from about 200 to 2000 nm, and the like. Depending on the operation, heating and/or cooling may be desired, as illustrated by thermal cycling with PCR.

A. Nucleic acid reaction methods

Several types of nucleic acid reactions can be carried out with the device of the invention. By having a main trench or channel, one has numerous sites with individual sources, so that at each site, the primers may be the same or different. A DNA sample is introduced into the main channel. The sample may be genomic DNA, a cDNA sample, a sample in which DNA fragments have been amplified using the polymerase chain reaction (PCR), genomic fragments, e.g. restriction endonuclease fragments, and other types of DNA sample material with a plurality of target sequences.

The sample is introduced to the site as single stranded DNA or may be denatured at the site, followed by reducing the temperature to provide for hybridization conditions. The hybridization medium is incubated for sufficient time for hybridization to occur between homologous or complementary sequences between the primer and the sample DNA, depending on the degree of stringency.

Where the device used does not contain feeder side channels (the FIG. 4 embodiment) the bulk phase medium added to the channel includes, in addition to the DNA or RNA sample material, common components required for the desired reaction, except for the reaction-specific oligo- or polynucleotides that will be provided in each reaction region. For example, for conducting simultaneous PCR reactions, the bulk-phase medium will contain, in addition to the DNA sample, a template-dependent polymerase, e.g., TAQ polymerase, all four deoxynucleotide triphosphates (dNTPs) and suitable salt and buffer components. In some instances one may have one, some or all four ddNTPs, or limiting concentration of some of the dNTPs, in the medium to provide termination at different nucleotide positions. Where the reaction is designed for primer extension, e.g., in DNA sequencing, the bulk-phase medium would contain mixtures of ddNTPs having a specific fluorescent species to designate each of the ddNTPs. Components employed in other nucleic acid reactions are considered below.

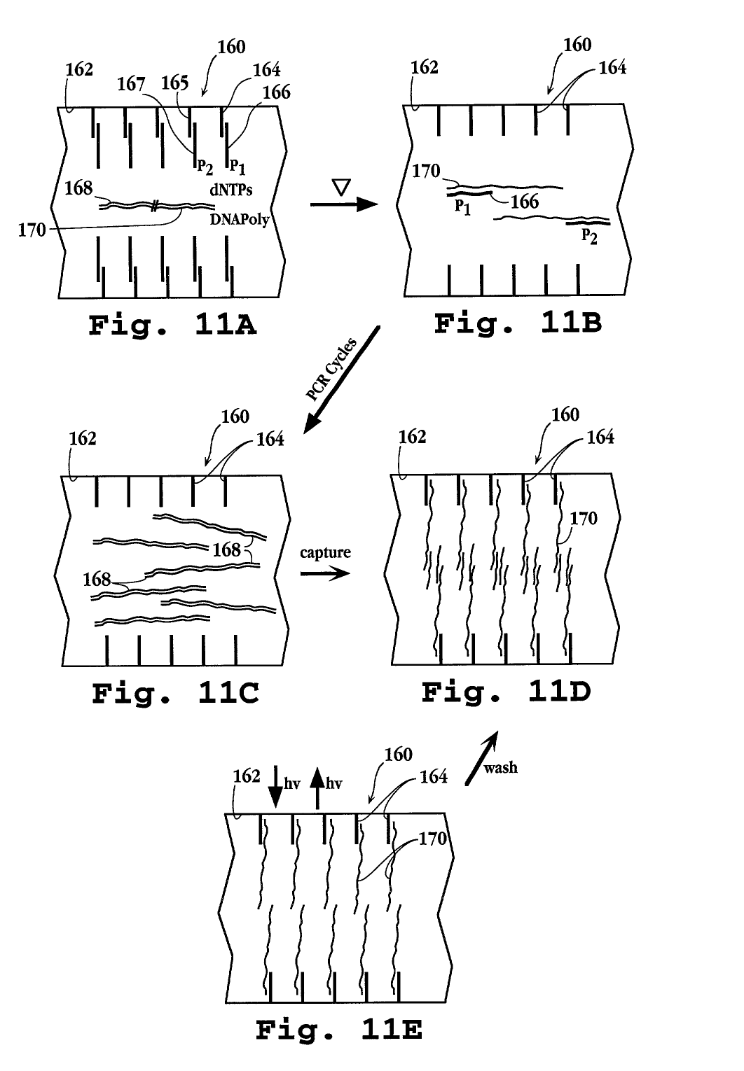

FIGS. 11A-11E illustrate steps involved in the use of the present invention for carrying out simultaneous PCR reactions. The figures show one reaction region 160 in a multi-channel device like the one shown in FIG. 1. The region has a wall portion 162 having covalently bound thereto, two different-sequence capture probes, 164, 165, which have sequences complementary to PCR primers 166, 167, respectively. The two primers (P₁ and P₂ in the figures) are reaction-specific PCR primers for a particular target DNA sequence, and are unique to reaction region 160. That is, different reaction regions includes a different set of PCR primers for amplifying a different target DBA sequence, it being recognized that some regions may have identical primers for control and sample duplicate purposes, or different quantities of the same primer sets.

A bulk-phase medium introduced into the device's channel includes double-stranded target DNA whose individual strands are indicated at 170. The bulk-phase medium also includes other PCR reaction components as noted above. The device is heated to DNA denaturing temperature, simultaneously denaturing the sample dsDNA and releasing primers P₁ and P₂ from the wall portion in each reaction region, as indicated in FIG. 11B, which also shows the primers annealed to the sample single strands after cooling under annealing conditions. The heating step typically is such as to raise the temperature of the bulk-phase medium to about 94° C. for a period of 1-5 minutes.

After a selected number of cycles of heating and cooling to effect denaturation, annealing and extension, the reaction mixture in each reaction region includes amplified sample dsDNA product or amplicon, as indicated at 168 in FIG. 11C, where the amplicon is different for different regions.

In one embodiment of the method, the bulk-phase medium is removed from the channel, yielding a mixture of all of the individual amplicons that can then be individually analyzed and/or isolated, e.g., by gel electrophoretic methods.

Alternatively, in a second embodiment, each channel can be employed as an electrophoretic separation channel, by applying a voltage potential across the channel ports, and detecting and/or isolating each amplicon as it migration past a detection and/or collection point adjacent one of the ports.

In a third embodiment, illustrated in FIG. 11D, the amplicons are partially purified by capture in single-stranded form on the capture probes in each reaction regions, by a capture heating and cooling step, and flushing the channel to remove unbound material. It will be appreciated that in this embodiment, the capture probes must contain sequence complementary to a sequence in the amplicons, and preferably to each amplicon strand.

In a fourth embodiment, illustrated in FIGS. 11D and 11E, the amplicons are both captured within each associated reaction region, and analyzed in situ in captured form. In this embodiment, the PCR reaction is carried out in the presence of detectable probes, such as fluorescently labeled nucleotides. The amplicon strands are optionally captured on the capture nucleic acids, and analyzed in situ, e.g., by examining each reaction region successively with a fluorescence scanner or microscope, to determine the presence and/or qualitative amount of fluorescence present in each reaction region.

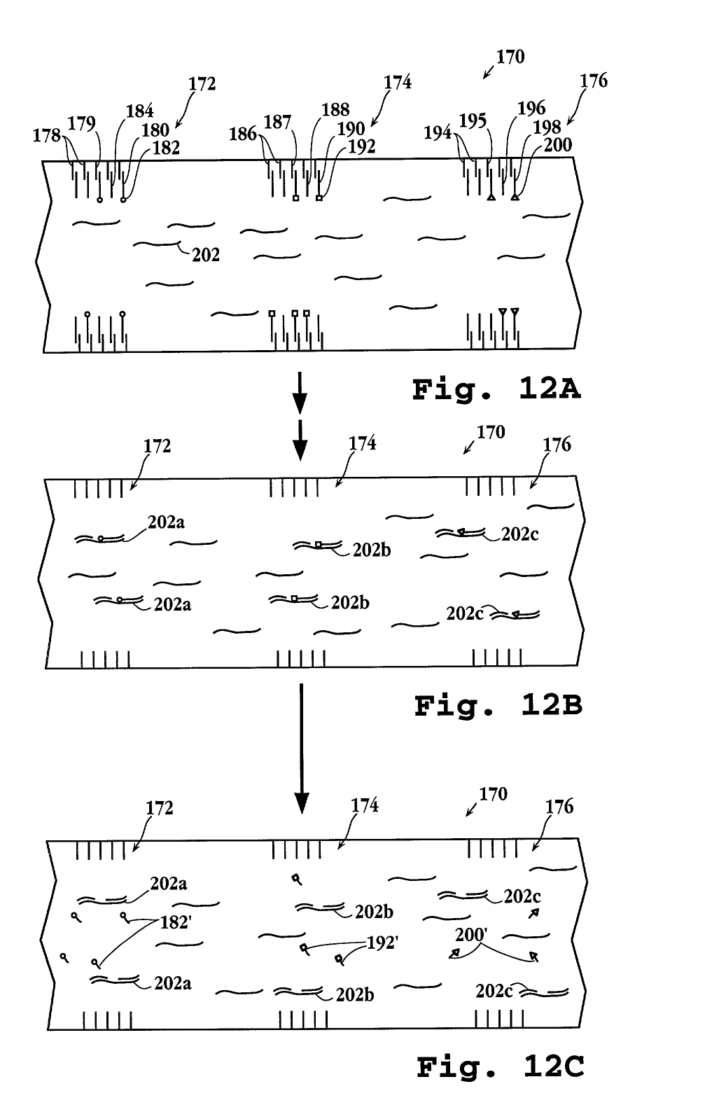

FIGS. 12A-12C illustrate a sequence analysis method that is advantageously carried out in accordance with the present invention. The method employs DNA primers having 5′-end electrophoretic tags that having (i) unique electrophoretic mobilities, by virtue of unique charge/mass ratio, and (ii) detectable moieties, such as fluorescent groups. Such tags are detailed, for example, in co-owned patent applications Ser. No. 09/303,029, filed Apr. 30, 1999, Ser. No. 09/561,579, filed Apr. 28, 2000 and corresponding PCT application PCT US00/10501, Ser. No. 09/602,586, filed Jun. 21, 2000 and Ser. No. 09/684,386, filed Oct. 4, 2000, all of which are incorporated herein by reference.

The figures show three reaction regions 172, 174, 176 in a multi-reaction device 170. Each reaction region contain two different-sequence immobilized capture probes, such as probes 178, 179 in region 172, probes 187, 186 in region 174 and probes 194, 195 in region 176. In the particular method to be described, for detecting single base mutations, such as snps, in target DNA, the oligonucleotide reagents that are carried on and released from the capture probes include an unlabeled upstream primer, which is designed to bind the target DNA upstream of the site of mutation, whose binding to the target site is determined by the presence or absence of the potential mutation. The upstream primers include primer 184 in region 172, primer 188 in region 174, and primer 196 in region 176. The site-specific primer includes a detectable electrophoretic tag, such as described and referenced above, that can be used to provide a characteristic electrophoretic signature of that primer. In the figure, the site-specific primers and their detectable tags are indicated respectively at 180, 182 in region 172; at 190, 192, in region 174; and at 198, 200 in region 176.

In operation a bulk phase medium containing a plurality of target DNA 202, and a DNA polymerase with 5′-exonuclease activity is added to the device channel, bringing the target DNA and other bulk-phase reaction components, e.g., all five dNTPs, into each of the reaction regions, as illustrated in FIG. 12A. The device is then heated, or otherwise treated to release the two primers in each reaction region, and subsequently cooled, as above, to anneal the primers to upstream and mutations sites on region-specific target sites. The step is illustrated in FIG. 12B, where target DNA strands 202, 204, and 206 in the three regions indicate different target sequence that are complementary to the primers in the three different regions. In particular, the upstream primer will hybridize to a region upstream of a potential mutation in the specific target region, and the extent of binding of the site-specific primer to the mutation site target area will be influenced by the presence or absence of a particular base at the mutation site.

After primer binding to the respective target regions, the action of the polymerase enzyme begins to extend the upstream primer until the growing chain reaches the site-specific mutation. Depending on the presence or absence of a given base at the potential mutation site, the enzyme will cleave the electrophoretic tag from the site-specific primer, releasing it from the target/primer dsDNA, as indicated in FIG. 12C.

The bulk-phase medium may now be removed from the channel, as above, and the electrophoretic tags detected and identified by electrophoresis, thus to identify particular mutations contained in the target DNA. As above, the reaction products, particular cleaved and uncleaved site-specific primer sequences, can be recaptured within each reaction site, to remove such sequences from the bulk-phase sample before analysis.

In a variation of the method, the release of tags from the site-specific primers will be detected by (i) capturing all of the cleaved and uncleaved primer on the reaction-site wall portion, (ii) applying a potential difference across the two channel ports and (iii) sequentially detecting tags as they pass through a detection zone near the downstream end of the channel. In this method, the tags from the different primers will all have the same electrophoretic mobilities, so that the presence or absence of a tag in any reaction region can be determined by the absolute migration times of each detected tags, or the relative migration times of adjacent tags.

The method and device provide a number of advantages in carrying out simultaneous reactions involving nucleic acid targets. For carrying out simultaneous PCR reactions, the method minimizes the possibility of specious amplification products formed by mismatched primers, since each reaction is carried out substantially in the presence of one primer set only. The reaction in each region can be carried out to higher amplicon levels, since the concentration of a single primer pair in each region can be relatively high. Finally, the amplicon products can be detected directly in isolated form, by capture of labeled amplicon strands on the wall portion of each reaction region.

Similar advantages apply to DNA extension methods, such as the one described with respect to FIGS. 12A-12C. The possibility of false positives, due to primer mismatches, is substantially reduced because only a single primer pair is present in each reaction region (or only a single primer pair is present at high concentration, considering the possibility of some primer diffusion from adjacent reaction sites). The amount of signal produced can be enhanced, because of the greater concentration of a single primer or primer set in each reaction region. Finally, the reaction products can be detected in situ, by electrophoresis of reaction products through the device channel, or by analyzing individual reaction components in the bulk-phase solution.

The variation in reaction protocols can be expanded in a device like that of FIG. 4 having side channels feeding each reaction region in a device. For example, excess soluble primer sequence (unable to bind at the site to the surface) may be added under mildly denaturing conditions to displace the primer from the wall portion. Reaction products, such as labeled DNA or duplex DNA can be diverted from the channel directly into a side channel for detection in a side-channel reservoir. Restriction endonuclease or other site-specific reagents may also be introduced into the individual reaction regions in this embodiment of the device.

B. Affinity determinations with a side channel device

In the embodiment illustrated in FIG. 4, having a pair of side channels associated with each reaction region, the channels may provide a source and drain, so that agents may be moved across the site in accordance with the needs of the operation. The agents may include reagents, washing solutions, or other agents associated with the operation. Operations may include DNA sequencing, DNA characterization, competitive and non-competitive binding assays, homogeneous and non-homogeneous assays (where the distinction is whether there is a separation step involving washing away unreacted label or not). The solutions may be moved by any convenient means, including electrokinetic, particularly electroosmotic, pneumatic, e.g. pumping, hydraulic, piezoelectric, sonic, etc. The particular choice will depend upon convenience, the precision with which the solution must be metered, the volume of solution, the nature of the equipment, i.e. the capabilities of the equipment, and the like.

Assays that may be performed may be homogeneous (no separation step) or heterogeneous, requiring a separation step, although the detection may be at the channel site or a distal site. Assays may involve labels such as light emitting detectable labels, e.g. fluorescers, chemiluminescers, energy transfer labels involving two different dyes at a distance which results in energy transfer upon irradiation of one dye and emission of the other dye, lanthanide dyes, which provide time delayed emission, where the lanthanide dyes may be used in particles, since they do not result in significant energy transfer or quenching, etc., enzymes, where the substrate results in a detectable product, which can be a dye, fluorescer, radioisotope, particle, etc., radioisotope, particle, e.g. colloidal carbon, colloidal gold, latex, etc., and the like.

For the heterogeneous assays, the protocols may involve release of the detectable label, so that the detectable label is assayed distal from the channel site. As illustrative of an assay would be the determination of a protease. By having a detectable label bound to the surface by a chain having a recognition sequence for the protease, one can monitor compounds modulating the activity of the protease. One may bind the detectable label through the proteolytically hydrolysable group to the surface at the site. One would premix the enzyme and the candidate compound to allow for binding of the two components. The mixture would then be moved through a lateral branch channel to the main channel site and allowed to incubate, ensuring that any additional reagents necessary for the proteolysis were present. After sufficient time for reaction to occur, the mixture at the main channel site would be moved into a lateral branch channel for detection of the label. The signal observed would then be related to the effect of the candidate compound on the enzyme activity. Rather than a candidate compound, there may be instances when one is interested in the enzyme activity of a cell. In this case a lysate could be prepared, where the enzyme of interest may be further processed to remove debris, other proteins, e.g. using HPLC, an affinity column, etc., and then moved through the lateral branch channel to the main channel site. Again, one could measure the activity of the enzyme in the lysate. Usually, one or more control may be performed in the same way as the assay, for comparison of the result from the sample.

There are numerous protocols for enzyme assays, depending upon the nature of the enzyme and the information desired. For example, one may be interested in a protease and/or proenzyme, where the protease activates the proenzyme. By binding the protease at the main channel site, one can add a sample suspected of containing the proenzyme to the main channel site and incubate for sufficient time for any proenzyme to be activated. One would then add substrate for the activated enzyme, where the product of the substrate can be detected. A similar assay could be to detect an enzyme requiring a coenzyme to form a holoenzyme.

Other assays may involve ligand-receptor binding, which may be competitive or non-competitive. In the competitive mode, a labeled ligand biomimetic would be non-covalently bound to the receptor, which in turn would be bound to the channel site. The ligand competitor would be moved to the channel site and allowed to incubate, where the degree of displacement of the labeled biomimetic would depend on the binding affinity of the ligand competitor. The binding affinity may be determined using kinetic or equilibrium measurement. This assay can be carried out homogeneously, where binding of the biomimetic to the receptor affects the signal, for example, fluorescence polarization or quenching. Quenching may be as a result of the interaction between the receptor and the label or the presence of a quencher bound to the receptor. By reading the change in fluorescence, one can determine the binding affinity of the ligand competitor. At completion of the assay, one would wash the site free of the released biomimetic ligand and the ligand competitor and then replenish the labeled biomimetic through the lateral channels. After washing any excess biomimetic ligand from the channel site, the channel site would be ready for the next assay.

In other assays one may use particles that provide for detection when the particles are in close proximity. One may use the LOCI technology, where one particle has a catalyst for forming singlet oxygen from hydrogen peroxide and the other particle has a dye that provides a detectable signal upon reaction with singlet oxygen. See, for example, U.S. Pat. Nos. 5,545,834 and 5,672,478. By having one of the pair of particles fixed at the main channel site and the other in solution, when the particles are brought together at the main channel site, in the presence of the other reagents, a signal will result. The effect of a candidate compound on the degree to which the particles are brought together can be a measure of the activity of the candidate compound. In any combination of two components that have a specific affinity for each other, the signal will be related to the degree to which the candidate compound interferes with the binding. Thus, one may interested in ligand receptor binding, whether a naturally occurring protein or candidate compound interferes with or augments complex formation between two proteins, the presence of a component in a sample in a diagnostic assay, where the component may be a drug, pollutant, pesticide, process contaminant, etc.

The assay would be performed by mixing the soluble particle with the compound to be assayed and moving the mixture with the additional reagents to the main channel site. The mixture would be incubated to allow for binding to occur and the signal read. The resulting signal could be compared with a control to determine the activity of the compound being assayed. As before, at completion of the reaction, the site could be washed free of all of the spent and unspent reagents and the process repeated.

Where one is interested in a polyepitopic compound, one has the opportunity to use non-competitive binding. For detecting the presence of a polyepitopic compound, one could use an ELISA assay, employing two antibodies: a bound antibody and a labeled antibody, where the two antibodies bind at different epitopes of the compound. One would add the compound through a lateral channel to the main channel site and incubate to allow for binding. One would then pass a wash solution through to remove non-specifically bound components of the sample, followed by addition of the labeled antibody. After washing away unbound labeled antibody, one would then detect the label present at the main channel site. The subject methodology may also be used to enrich a mixture for a desired component by providing for capillary electrophoresis in the source lateral channel, where the desired component would be concentrated when encountering the site. The remaining components could be washed through the site, where non-specific binding components would not be retained in the main channel, but directed to a waste channel.

The device may have independent source and waste reservoirs, or may have a connecting channel between multiple source and/or waste reservoirs, so that solutions may be added or withdrawn simultaneously from a plurality of reservoirs, may have crossed channels at the source for precise injection of volumes into the main channel, may have a plurality of reservoirs feeding into the source channel or receiving waste from the waste channel, etc. The waste channel may have a detector, providing means for irradiation of the waste channel and detection of light emission or absorption, or there may be a channel independent of the waste channel or incorporating a portion of the waste channel that serves as a detection channel. The solutions may be moved in any convenient way, pneumatically—positive or negative pressure, electrokinetically—electrophoretically or electro-osmotically, hydraulically, or the like. The choice will be based on accuracy, nature of the operation equipment available, sensitivity to variations in volumes, etc. Therefore, the reservoirs will be fitted with the necessary devices to provide for liquid movement.

The following examples are offered by way of illustration and not by way of limitation.

### EXAMPLE 1

### Preparation of a Channel Region with Primers and Performing PCR

A. Synthesis of SPDP-BSA-benzophenone

First, mix 10 mg each of succinimidyl-3-(2-pyridylthiopropionate) (SPDP) and 4-benzoylbenzoic acid in a brown bottle and dissolve the mixture in 1 mL anhydrous dimethylformamide. Next, dissolve 100 mg bovine serum albumin (BSA) in 6 mL phosphate buffered saline, pH 7.2. Combine the two solutions by adding ˜50 uL of the DMF solution to the solution of BSA, with vortexing, every 30 minutes. Keep the reaction solution in the dark, agitate the reaction solution on a shaker (150 rpm) between additions. After the additions are complete continue shaking the solution at room temperature until 2 days have elapsed. Then, dialyze the reaction against water for 1 day, in the dark, with three changes of water. Centrifuge the solution for 10 min at 3000 rpm, and collect the supernatant. Lyophilize the supernatant to dryness, and store the product, SPDP-BSA-benzophenone, as a solid at −20C. For use in experiments, prepare a 10 mg/mL solution of SPDP-BSA-benzophenone in 1×PBS buffer (pH 7.2).

B. Surface attachment of SPDP-BSA-benzophenone in a channel and formation of devices with region-specific capture nucleic acids

A polycarbonate substrate with channels 50 um deep, 120 um wide and 50 mm long was prepared by compression molding. The surface of the plastic substrate was washed with water, dried with a tissue, and ˜30 uL of the 10 mg/mL solution of SPDP-BSA-benzophenone was pipetted into the channel. A rubber gasket was placed on the surface of the substrate surrounding the channel, and on top of the gasket was placed a mask prepared with black electrical tape and a glass slide. A portion of the tape was cut out to provide irradiation to a 3 mm long section of the channel. Another slide was positioned under the substrate for support, and the 4-layer assembly (mask, gasket, substrate, support slide) was clamped tightly. The assembly was exposed for 20 min to a collimated beam of light from a 100W mercury arc lamp. After disassembly the substrate was washed three times each with 0.05% Triton X-100 and water. The channel was thus prepared with a region carrying an activated disulfide bond-forming group, where the region was defined through masked photodeposition of the light-sensitive reagent. A capture nucleic acid, 1, having a terminal thiol group was prepared, and 250 pmol were dissolved in 50 uL of 0.5M carbonate buffer. A portion of the solution was pipetted into the channel at the irradiated region, and incubated at room temperature for 2 hr. The substrate was then washed with water, dried, and the open channels of the substrate were sealed by thermal lamination with a 40 um thick film of PMMA (MT-40).

C. PCR

PCR primers 2 and 3, targeting the beta-actin gene, were prepared with a target specific 3′ end portion, a 5′ end portion complementary to the sequence of the capture nucleic acid 1, and a non-amplifiable polyoxyethylene spacer moiety linking the two portions. The primers were combined in a PCR reaction mix consisting of 1×PCR buffer II, 200 uM TTP, 200 uM dCTP, 200 uM dGTP, 40 uM dATP, 160 uM F-dATP (fluoresceinated dATP), 1.5 mM MgCl₂, 0.01% BSA, 0.5 uM primers 2 and 3, and optionally a diluted sample of an unlabeled product solution of the beta-actin amplicon as template. The PCR mix was added to the channels prepared as above. Samples with and without the template were prepared. The reservoirs were taped closed, and the substrates were placed on an MJ Research thermocycler unit with a flat block and thermocycled according to the protocol: denature at 92C for 2 min; 26 cycles of 92C for 1 min, 54C for 1 min, and 72C for 30 sec; final extension at 72C for 5 min, and hold at 4C until retrieved. The reaction was also performed in a standard PCR tube as a control.

After the reaction was complete the solution was removed from the channels and tubes and analyzed by polyacrylamide gel electrophoresis. Also, the channels were refilled with TENSS buffer (100 mM Tris, 25 mM EDTA, 300 mM NaCl, 0.1% dextran, 0.01% salmon sperm DNA) and the channels examined by fluorescence microscopy. PAGE analysis revealed the presence and absence of product amplicon bands where the reaction was carried out with and without template, respectively. The image analysis showed that the irradiated region of the channel treated with SPDP-BSA-benzophenone gave a strong fluorescent signal after thermocycling the reaction mix containing the template whereas the non-irradiated regions yielded no signal. No fluorescence was observed in the treated channels when the template was not in the reaction mix. Such results indicate that the reaction produced amplicons with fluorescent labels incorporated in the strand, and the amplicons, generated with single-stranded ends because of the non-replicable moiety, hybridized to the capture nucleic acids immobilized on the surface of the channel.

### EXAMPLE 2

### Preparation of a Channel Region with Capture Nucleic Acids and Measurement of the Binding Capacity

A. Synthesis of biotin-BSA-benzophenone

The procedure for preparing biotin-BSA-benzophenone was the same as that given above for the preparation of SPDP-BSA-benzophenone, replacing SPDP with (biotinylamidocaproylamido)caproic acid N-hydroxysuccinimide (Biotin-X-X-NHS).

B. Preparation of streptavidin-coated channels

First, biotin-BSA-benzophenone was attached to channel surfaces by the same methods as described above for SPDP-BSA-benzophenone. After irradiation and washing away unbound materials, a 0.1% solution of streptavidin in TE buffer, pH 8.0 was added to the channel and incubated at room temperature for 30 min. The channel was then washed three times each with 0.05% Triton X-100 and water. The substrate was then dried, and the open channels of the substrate were sealed by thermal lamination with a 40 um thick film of PMMA (MT-40).

C. Demonstration of the formation of reaction-specific reagent regions

An oligonucleotide duplex was prepared using one biotinylated oligo, 4, and one fluorescein-labeled oligo, 5. Equimolar solutions of 4 and 5 were combined in TENSS buffer with a final concentration of 10 uM. To ensure formation of the duplex, the solution was heated to 70C for 15 min and then left to cool at room temperature for 30 min prior to use. This stock solution was further diluted to 1 uM concentration and introduced into the treated channel. After 10 min incubation, the solution was removed and the channel rinsed with 0.5 mM MgCl₂, 50 mM Tris [pH 8.0] buffer. Imaging the channel by fluorescence microscopy revealed a fluorescent signal in the region of the channel that was irradiated through the mask. Irradiation effected the deposition of biotin-BSA-benzophenone, which in turn bound the streptavidin to this region. The oligo duplex binded to this region via complex formation between the biotinylated oligo and the surface streptavidin, which yielded the signal due to the labeled oligo hybridized to the biotinylated oligo.

To confirm the nature of this localization of the duplex, a competitor oligo, 6, was added at a concentration of 50 uM in 0.5 mM MgCl₂, 50 mM Tris [pH 8.0] buffer to the channel. The fluorescent signal disappeared within minutes. The sequence of 6 was designed as a competitor to oligo 5, having a longer region that is complementary to the capture oligo 4 and thus able to cause the displacement of oligo 5.

D. Measuring the surface binding capacity

The surface binding capacity of a surface treatment for the carrying of reaction-specific reagents determines the solution concentration of these reagents when released for the performing of a reaction, or the surface concentration of a heterogeneous reagent employed in immobilized form. The surface binding capacity of channels treated with biotin-BSA-benzophenone and streptavidin was determined by two methods. In one, duplexes of 4 and 5 were preformed, bound to the surface, and the amount of fluorescent signal released from the channel upon addition of the competitor 6 was quantified. In the second, the capture oligo 4 was first bound in the channel to create a channel surface carrying one member of a specific binding pair. Then the reagent 5 was added to the channel, where the two oligo binding pair members formed the duplex. Again, the competitor was added to cause the release of the labeled oligo, which was collected and quantified. The released solutions were brought to the same volume using the buffer solution, and a series of solutions of known concentration of the labeled oligo were used to prepare a standard curve relating fluorescence intensity to the amount (or concentration) of fluorophore. The results indicated that the same binding capacities were obtained by either method of preparing the channel. The binding capacity varied with the concentration of 5 introduced into the channel, increasing to a binding capacity of about 0.08 pmol/mm² as the concentration of 5 reached 1.5 uM.

### EXAMPLE 3

### Fabrication of Regions of Reaction-specific Reagents

As in Example 2C, reaction-specific reagent regions were prepared. In Example 2C, the first layer in the structure was defined by the irradiation pattern and subsequent layers conformed to this spatial definition, whereas, in this experiment the underlying layers were prepared uniformly along the channel and the localized reagent regions were defined by the localized delivery of reagents to the treated surface. Channels were prepared in polycarbonate substrates by either milling or compression molding. The channels were washed with soap and rinsed with MilliQ water. A 1% solution of biotin-BSA-benzophenone was pipetted into the channels and irradiated for 15 min with a 100W mercury arc lamp through a glass slide filter. The channels were then rinsed three times each with 0.05% Triton X-100 and deionized water and then dried. The channels were then treated with a 0.1% solution of streptavidin. After incubating at room temperature for 30 min, the channels were rinsed three times with 1×PBS solution, or alternatively a 1×PBS, 1% BSA solution. Another duplex of one biotinylated oligo, 7, and one fluorescein-labeled oligo, 8, was prepared in TENSS buffer to a final concentration of 1 uM, annealed as described above, and spotted in the channels to define regions of various lengths and number in a series of channels. The channels were rinsed of the excess, unbound materials by washing three times with 0.5 mM MgCl₂, 50 mM Tris [pH 8.0] buffer. The washes were collected and measured for fluorescence. The results demonstrate that by the final wash no more fluorscent signal is being recovered from the channel. The fluorescein labeled oligo was then stripped off the channel surfaces by adding a denaturing release solution of 70% aqueous formamide. A standard curve relating fluorescence intensity to amount of labeled oligo was prepared using a series of dilutions of known concentration of the labeled oligo. Control experiments demonstrated that the release solution does not cause release of the capture oligo 7. The series of regions created and quantified are summarized in the table below.

| | | | | | | | |
|---|---|---|---|---|---|---|---|
| No. of regions | 1 | 5 | 4 | 3 | 2 | 2 | 2 |
| Length of region | 18 | 2 | 2 | 2 | 2 | 3 | 4 |
| Total effective length | 18 | 10 | 8 | 6 | 4 | 6 | 8 |
| Signal of released | 3.29 | 1.47 | 0.85 | 0.63 | 0.42 | 0.94 | 0.88 |
| oligo | | | | | | | |

The relationship thus determined between the surface capacity and the size and number of regions carrying nucleic acid reagents demonstrates the ability to fabricate such regions having a useful amount of reagent for reactions.

### EXAMPLE 4

### Multiplex PCR Using Devices of the Subject Invention with Primers Releasably Bound via Hybridization

Channels were prepared in polycarbonate substrates of dimension 0.4×0.8 ×18 mm. Ports were made by drilling holes at the channel ends to the opposite surface, and the channel was enclosed by laminating a thin polycarbonate film to the side of the substrate with the open channels. The surface of the channel was treated as described in Example 3 with biotin-BSA-benzophenone and streptavidin. Then, primer sets were introduced into separate regions by incubating solutions of primer/capture nucleic acid duplexes in distinct portions of the channels. A 3-plex reaction using three the primer pairs was performed in a channel device with each primer set localized to separate regions, and the three combinations of 2-plex reactions were also performed with each primer set localized to separate regions. For use in the device, each primer of a pair was prepared with the same 20-mer capture sequence extending from the 5′ end of the primer sequence, and a capture probe was prepared with the complementary capture sequence and a 3′ biotin. Thus, capture nucleic acid 9 for primers 10 and 11; capture nucleic acid 12 for primers 13 and 14; and capture nucleic acid 7 for primers 15 and 16. Each set was prepared in duplex form using a molar ratio of 2:1:1 of capture nucleic acid:primer:primer, in TENSS buffer, annealed as described above. Each set was introduced separately into the channel and incubated for 30 min. To prepare the 2-plex reactions in localized regions each primer set was introduced via each of the two terminal ports with the solutions only filling around half the channel so as not to permit mixing. For the localized 3-plex reaction, two sets were introduced again via the two terminal ports to only one-third the channel length. After these binding reactions, the third set set was introduced to the middle region for binding to the remaining free sites in that region. After the primer sets incubations the channels were rinsed with 1×PBS. The homogenous 3-plex reaction was also performed in tubes and in the channels. In the case of the channels the primers were supplied with the PCR reaction mix, but the surfaces were treated with biotin-BSA-benzophenone and streptavidin, though no capture nucleic acids were added.

After loading the channels with the bound primer reagents, the channels were filled with a standard PCR mix. The loaded plastic device was sealed with pressure sensitive adhesive (PSA) film, placed on a PE 9700 thermocycler instrumet (the channels were designed to fall on top of the metal surface and not on top of the holes for holding tubes) with a plastic shim on top of the device, and secured in place by closing the lid. The shim acted to transfer the pressure of the lid down to the PSA film. The thermocycler was programmed as follows: 94C, 10 min; 35 cycles of 94° C., 45 sec; 58° C., 30 sec,; 70C, 45 sec; with a final extension at 70C for 10 min. Following the reaction the solutions were removed from the channels and analyzed by 2% agarose gel electrophoresis.

This homogenous 3-plex reaction failed to produce one of the three amplicons in significant amounts in all the samples run. However, with the primers localized to separate regions in the channel, in both the cases of 2-plex and 3-plex reactions, the reactions proceeded to yield all the expected amplicon products in the expected amounts as determined by gel analysis.

The results of this experiment demonstrate that within one fluidly connected channel, localized primer sets can react in combination with the same common reagents provided in the bulk. Furthermore, as observed with the 3-plex reaction, starting with spatially separated reaction-specific reagents, wherein the reactions proceed in substantial isolation may provide a better yield of the different products than when performed as a typical multiplex wherein the reagents are fully mixed.

### EXAMPLE 5

### Multiplex PCR Using Devices of the Subject Invention with Primers Releasably Bound via Ligand Binding and a Demonstration of the Substantial Isolation of Reaction Regions

Devices as described in Example 4 were again fabricated and prepared with biotin-BSA-benzophenone and streptavidin treated surfaces. Two primer pairs, 17, 18 and 19, 20, were prepared with biotinylated 5′ ends. Solutions of various combinations of the primers were prepared in 1×TE buffer, and introduced into the channel and incubated for 30 min at room temperature to establish channel surfaces with primers bound via the biotin/streptavidin linkages. The primer combination prepared were as follows: set A (17, 18, 19, 20); set B (17, 18); set C (19, 20); set D (17, 19); set E (18, 20). Sets A, B and C are proper combinations of primers in that they yield amplified products, with set A being a 2-plex reaction which was established to consistently produce both amplicons well. Sets D and E however are improper combinations that in isolation do not yield any amplified products. Channels were prepared in duplicate in the following manner: 1: set A; 2: sets B and C in separate regions of the channel with no gap between the regions; 3: sets B and C in separate regions of the channel with a 1 mm gap between regions; 4: sets D and E in separate regions with no gap between the regions; and 5: sets D and E in separate regions with a 1 mm gap between regions. After incubating the primer solutions in the channels the channels were again rinsed with 1×PBS. As in example 4, the PCR reaction was introduced into the channels, the ports were sealed with PSA film, the device and shim secured in the thermocycler, and the reaction performed using the same cycling protocol listed above. The solutions were removed after cycling, combined with a loading buffer and analyzed by 2% agarose gel electrophoresis.

The 2-plex reaction of 1 produced the expected two bands of the two amplicons, and the reactions of 2 and 3, with the two different primer pairs in separate regions also produced the same two bands in similar yields. Reaction 4 produced significantly less product of each of the two amplicons, with bands discernible by eye but too feint to accurately quantify. Reaction 5 failed to produce any visible bands. Each reaction was performed in duplicate, and gave identical results.

The results of this experiment demonstrate the utility of using directed binding of ligand-labeled (biotinylated) primers to receptor-bearing (streptavidin) surfaces for establishing defined regions of different reaction-specific reagents. This and other experiments have also demonstrated that primers bound via ligand/receptor complexes are released from the surface into solution in the course of the thermal cycling protocol by thermal denaturation of the complex. Also, by setting up wrong primer combinations of a functional 2-plex set, this experiment also demonstrates that convective mixing is not occuring on the time-scale of the reaction and thus the reactions are regionalized according to the placement of the reagents and proceed in substantial isolation.

### EXAMPLE 6

### Amplification using Secondary Primers

Devices as described in Example 4 were again fabricated and prepared with biotin-BSA-benzophenone and streptavidin-treated surfaces. Capture nucleic acid/primer pair, (probe 7/primers 15, 16 and probe 12/primers 13, 14) solutions were separately prepared in TENSS solution as described above, and separately introduced into a series of channels, and incubated for 30 min. After rinsing the channels of the unbound probes, a standard PCR reaction mix was added, with varying amounts, concentrations of 0, 0.1, 0.3 and 0.5 uM, of a corresponding secondary primer (primer 21 and 22, respectively) in the mix. The secondary primers have the same sequence as the capture sequence portion of the primers. The devices were sealed and thermocycled as previously described. The reaction products were analyzed by agarose gel electrophoresis.

The reactions each produced the expected amplicon product. The amount of product however increased with increasing concentration of the secondary primer, ultimately yielding approximately 100% more product when present at 0.5 uM concentration as determined by the band intensities for both primer sets. Separate control experiments lacking the primary primers, 15 and 16, or 13 and 14, failed to produce any products.

This experiment demonstrates the utility of secondary primers for boosting the PCR amplification yield.

### List of Sequences

SEQ ID 1
5′ thiol AAC AGC TAT GAG CAT GCG CCA GGG TTT TCC CAG TCA
CGA C 3′
NOTE: thiol = thiol modifier C6
Sequence Type: probe
SEQ ID 2
5′ F CCT GGC GCA TGG TCA TAG CT P TCA CCC ACA CTG TGC CCA
TCT ACG A
NOTE: F = 6-(fluorescein-5(6)-carboxamido)hexyl; P = hexaethyleneglycyl
Sequence Type: primer
SEQ ID 3
5′ F CCT GGC GCA TGG TCA TAG CT P CGC AAC CGC TCA TTG CC
NOTE: F = 6-(fluorescein-5(6)-carboxamido)hexyl; P = hexaethyleneglycyl
Sequence Type: primer
SEQ ID 4
5′ B AAC AGC TAT GAG CAT GCG CCA GGG TTT TCC CAG TCA CGA C
Sequence Type: probe
SEQ ID 5
5′ F CCT GGC GCA TGG TCA TAG CT
NOTE: F = 6-(fluorescein-5(6)-carboxamido)hexyl
Sequence Type: primer
SEQ ID 6
5′ GTC GTG ACT GGG AAA ACC CTG GCG CAT GGT CAT AGC TGT T
Sequence Type: probe
SEQ ID 7
5′ ACA TCG GAC GCA GTG GAC CTC ACG TCT ACA AGT CGC CTG
APB
NOTE: B = biotinTEG; P = triethyleneglycyl
Sequence Type: probe
SEQ ID 8
5′ AGG TCC ACT GCG TCC GAT GTP F
NOTE: 6-(fluorescein-5(6)-carboxamido)hexyl; P = hexaethyleneglycyl
Sequence Type: primer
SEQ ID 9
5′ CTG ATG CCG AGA GCT GCC AAG CCC ATA TAC GAT GCC TCG
APB
NOTE: B = biotinTEG; P = triethyleneglycyl
Sequence Type: probe
SEQ ID 10
5′ TTG GCA GCT CTC GGC ATC AGT CAT CCA TCA TCT TCG GCA
GAT TAA
Sequence Type: primer
SEQ ID 11
5′ TTG GCA GCT CTC GGC ATC AGC AGG CGG TAG AGT ATG CCA
AAT GAA AAT CA
Sequence Type: primer
SEQ ID 12
5′ GCT ATG CGA CCG ACC TAC CGT TTG AGC CAT CAC AGT CCA
CPB
NOTE: B = biotinTEG; P = triethyleneglycyl
Sequence Type: probe
SEQ ID 13
5′ ACG GTA GGT CGG TCG CAT AGC AAT AGG AGT ACC TGA GAT
GTA GCA GAA AT
Sequence Type: primer
SEQ ID 14
5′ CGG TAG GTC GGT CGC ATA GCC TGA CCT TAA GTT GTT CTT
CCA AAG CAG
Sequence Type: primer
SEQ ID 15
5′ AGG TCC ACT GCG TCC GAT GTC GTT GTT GCA TTT GTC TGT TTC
AGT TAC
Sequence Type: primer
SEQ ID 16
5′ AGG TCC ACT GCG TCC GAT GTA TCC ACT GGA GAT TTG TCT
GGT TGA G
Sequence Type: primer
SEQ ID 17
5′ B CCG GAT ACC CAG TTT CTC C
NOTE: B = biotinTEG
Sequence Type: primer
SEQ ID 18
5′ B TGG GTA CCC CAG AAA CAG TC
NOTE: B = biotinTEG
Sequence Type: primer
SEQ ID 19
5′ B TCC CCG TCC TCC TGC AT
NOTE: B = biotinTEG
Sequence Type: primer
SEQ ID 20
5′ B AGG AAG GCC TCA GTC AGG TCT
NOTE: B = biotinTEG
Sequence Type: primer
SEQ ID 21
5′ AGG TCC ACT GCG TCC GAT GT
Sequence Type: primer
SEQ ID 22
5′ CGG TAG GTC GGT CGC ATA GC
Sequence Type: primer

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A device for carrying out a plurality of different reactions in a single bulk-phase reaction medium, comprising:
means defining an elongate or planar channel and a port for introducing such bulk-phase medium into the channel,
a plurality of discrete reaction regions within the channel, and
a reaction-specific reagent releasably carried on a wall portion of each reaction region, for reacting in solution with one or more reagents in the bulk-phase medium, when such medium is introduced into the channel, to effect a selected solution-phase reaction in each region,
where the channel is dimensioned to substantially prevent convective fluid flow among the reaction regions during such reactions.

2. The device of claim 1, wherein said channel defining means defines a one-dimensional channel having a substantially uniform cross-section along its length, channel width and depth dimensions between about 20-800 microns, and the reaction regions are submicroliter in volume.

3. The device of claim 1, wherein said channel defining means defines a channel having a plurality of radial bulges corresponding to the reaction regions, and connected in series by channel sections having channel width and depth dimensions between about 20-800 microns.

4. The device of claim 1, wherein said channel-defining means includes a pair of planar expanses that are separated from one another by a dimension between about 20-800 microns, and the reaction regions are submicroliter in volume.

5. The device of claim 1, for carrying out sequence-specific nucleic acid reactions involving target nucleic acid present in the bulk-phase medium, wherein the reaction-specific reagents are nucleic acid oligomer reagents releasably bound to the wall portions through duplex formation with immobilized complementary-sequence oligonucleotides, or via ligand attachment to an immobilized antiligand.

6. The device of claim 6, wherein each reaction region includes a capture nucleic acid immobilized on the associated wall portion and having a region-specific nucleic acid sequence, and wherein different-sequence nucleic acid oligomer reagents are hybridized with such capture nucleic acids.

7. The device of claim 6, for carrying out sequence-specific nucleic acid reactions selected from the group consisting of:
(a) polymerase extension reactions, wherein the reaction-specific reagents in each region include extension primers;
(b) PCR reactions in the reaction regions, wherein the reaction-specific reagents in each region include one or more sets of PCR primers.
(c) sequence-specific 5′ exonuclease reactions that result in the formation of a detectable product, wherein the reaction-specific reagent in each region include as an exonuclease substrate, an oligonucleotide having a selected nucleic acid sequence terminating in a detectably labeled 5′ nucleotide.

8. The device of claim 8, for use in carrying out 5′ exonuclease reactions, wherein detectably labeled 5′ nucleotides associated with different reaction regions are electrophoretically separable.

9. A device for carrying out simultaneous sequence-specific nucleic acid reactions on a plurality of DNA target segments (i) contained in a bulk-phase medium and (ii) having different nucleic acid sequences, comprising:
a substrate defining an elongate channel terminating at first and second ends,
a lid covering the open channel to form an elongate closed channel terminating at first and second ports,
a plurality of discrete reaction regions spaced along the length of said channel, between said ports, and
in each reaction region, one or more region-specific nucleic acids releasably carried on a portion of that reaction region,
where the region-specific nucleic acids are effective to bind to complementary sequence nucleic acid target segments contained in the bulk-phase medium, after such medium is introduced into the channel,
and the channel design substantially prevents convective fluid flow among the reaction regions in the channel,
whereby the region-specific nucleic acids are largely confined to the associated region during such reaction.

10. The device of claim 10, wherein each reaction region includes a capture nucleic acid immobilized on the associated wall portion and having a region-specific nucleic acid sequence, and wherein different-sequence nucleic acid oligomer reagents are hybridized with such capture nucleic acids.

11. The device of claim 10, for or carrying out sequence-specific nucleic acid reactions selected from the group consisting of:
(a) polymerase extension reactions, wherein the reaction-specific reagents in each region include extension primers;
(b) PCR reactions in the reaction regions, wherein the reaction-specific reagents in each region include one or more sets of PCR primers,
(c) sequence-specific 5′ exonuclease reactions that result in the formation of a detectable product, wherein the reaction-specific reagent in each region include as an exonuclease substrate, an oligonucleotide having a selected nucleic acid sequence terminating in a detectably labeled 5′ nucleotide.

12. The device of claim 10, wherein said substrate is designed to be placed in a centrifugation apparatus, such that centrifugation of the device is effective to cause liquid medium introduced at one port to fill the channel, or liquid medium contained within the channel to be expelled therefrom.

13. A method for simultaneously carrying out a plurality of different reactions that involve both common and reaction-specific reagents, comprising:
filling a channel having (i) means defining an elongate or planar channel and a port for introducing a liquid medium into the channel, and (ii) a reaction-specific reagent releasably carried on a wall portion of each reaction region, for reacting in solution with one or more reagents in the bulk-phase medium, when such medium is introduced into the channel, to effect a selected solution-phase reaction in each region, and
by said filling, and with release of reaction-specific reagent from the wall portion in each reaction region, simultaneously promoting reactions involving reagents provided in the bulk phase and the reaction-specific reagents in each of the reaction regions.

14. The method of claim 14, wherein after the completion of said reactions, the medium is removed from the device for analysis or processing of the plurality of reaction products.

15. The method of claim 14, for carrying out simultaneous PCR reactions on a plurality of different DNA targets contained in the bulk-phase medium, wherein said reaction-specific reagents in the different reaction regions include PCR primers designed to hybridize with and amplify different, selected regions of the DNA targets.

16. The method of claim 15, wherein promoting said reaction includes successively heating and cooling the device, under conditions effective to produce PCR amplicons.

17. A method for carrying out a plurality of simultaneous sequence-specific nucleic acid reactions on a plurality of DNA target segments (i) contained in a bulk-phase medium and (ii) having different nucleic acid sequences, comprising
adding to device having (i) means defining an elongate channel and a port for introducing a liquid medium into the channel, and (ii) region-specific capture nucleic acids immobilized on channel wall portions at a plurality of discrete reaction regions contained within and along the length of the channel, a solution containing a plurality of different-sequence nucleic acid reagents, each having a capture portion effective to hybridize to one of the capture nucleic acids and a reaction portion effective to hybridize to one of the target DNA sequences in the bulk phase medium, under DNA hybridization conditions, thereby to localize selected nucleic acid reagents at selected reactions regions in the channel,
filling said channel with such bulk phase medium, and
simultaneously promoting reactions involving target segments contained in the bulk phase medium and such region-specific nucleic acid reagents, by causing release of the nucleic acid reagents from the associated reaction-region wall portions.

18. The method of claim 17, which further includes capturing reaction in each reaction region, by hybridization of reaction product to the immobilized capture nucleic acids.

19. A method for performing a plurality of affinity determinations to determine the biological activity of candidate compounds employing an elongated channel having a cross-section in the range of about 10 um² to about 4 mm² and a plurality of sites at which are non-diffusively bound a first component of said affinity determination, wherein each site is bordered by a source trench and a drain trench for moving components of said affinity determination to and away from said site, said affinity determination comprising the binding of a candidate compound to an enzyme and employing an enzyme substrate which results in a detectable product, said method comprising:
electrokinetically moving each of said candidate compounds from each of said source trenches to each of their respective sites and incubating the resulting mixture at each site,
adding said substrate to said main channel,
incubating the resulting mixture at each site, resulting in a detectable product, electrophoretically moving said detectable product from said site to said drain trench, and
detecting said detectable product separate from other components of said affinity determination as a measure of said affinity determination,
wherein the length of said site and the cross-section of said channel are chosen to have a reaction volume for said affinity determination of less than about 100 nL.


SEQUENCE LISTING (22 sequences)

SEQ ID NO: 1 - DNA, 40 bp, Artificial Sequence
  probe
aacagctatgaccatgcgccagggttttcccagtcacgac

SEQ ID NO: 2 - DNA, 45 bp, Artificial Sequence
  primer
cctggcgcatggtcatagcttcacccacactgtgcccatctacga

SEQ ID NO: 3 - DNA, 37 bp, Artificial Sequence
  primer
cctggcgcatggtcatagctcggaaccgctcattgcc

SEQ ID NO: 4 - DNA, 41 bp, Artificial Sequence
  probe
baacagctatgaccatgcgccagggttttcccagtcacgac

SEQ ID NO: 5 - DNA, 20 bp, Artificial Sequence
  primer
cctggcgcatggtcatagct

SEQ ID NO: 6 - DNA, 40 bp, Artificial Sequence
  probe
gtcgtgactgggaaaaccctggcgcatggtcatagctgtt

SEQ ID NO: 7 - DNA, 40 bp, Artificial Sequence
  probe
acatcggacgcagtggacctcacgtctacaagtcgcctga

SEQ ID NO: 8 - DNA, 20 bp, Artificial Sequence
  primer
aggtccactgcgtccgatgt

SEQ ID NO: 9 - DNA, 40 bp, Artificial Sequence
  probe
ctgatgccgagagctgccaagcccatatacgatgcctcga

SEQ ID NO: 10 - DNA, 45 bp, Artificial Sequence
  primer
ttggcagctctcggcatcagtcatccatcatcttcggcagattaa

SEQ ID NO: 11 - DNA, 50 bp, Artificial Sequence
  primer
ttggcagctctcggcatcagcaggcggtagagtatgccaaatgaaaatca

SEQ ID NO: 12 - DNA, 40 bp, Artificial Sequence
  probe
gctatgcgaccgacctaccgtttgagccatcacagtccac

SEQ ID NO: 13 - DNA, 50 bp, Artificial Sequence
  primer
acggtaggtcggtcgcatagcaataggagtacctgagatgtagcagaaat

SEQ ID NO: 14 - DNA, 48 bp, Artificial Sequence
  primer
cggtaggtcggtcgcatagcctgaccttaagttgttcttccaaagcag

SEQ ID NO: 15 - DNA, 48 bp, Artificial Sequence
  primer
aggtccactgcgtccgatgtcgttgttgcatttgtctgtttcagttac

SEQ ID NO: 16 - DNA, 46 bp, Artificial Sequence
  primer
aggtccactgcgtccgatgtatccactggagatttgtctgcttgag

SEQ ID NO: 17 - DNA, 19 bp, Artificial Sequence
  primer
ccggatacccagtttctcc

SEQ ID NO: 18 - DNA, 20 bp, Artificial Sequence
  primer
tgggtaccccagaaacagtc

SEQ ID NO: 19 - DNA, 17 bp, Artificial Sequence
  primer
tccccgtcctcctgcat

SEQ ID NO: 20 - DNA, 21 bp, Artificial Sequence
  primer
aggaaggcctcagtcaggtct

SEQ ID NO: 21 - DNA, 20 bp, Artificial Sequence
  primer
aggtccactgcgtccgatgt

SEQ ID NO: 22 - DNA, 20 bp, Artificial Sequence
  primer
cggtaggtcggtcgcatagc